(19) United States
(12) Patent Application Publication
(10) Pub. No.: US 2025/0296985 A1
(43) Pub. Date: Sep. 25, 2025

(54) POTENTLY NEUTRALIZING NOVEL HUMAN MONOCLONAL ANTIBODIES AGAINST SARS-COV-2 (COVID-19)

(71) Applicants: TRANSLATIONAL HEALTH SCIENCE AND TECHNOLOGY INSTITUTE, Faridabad (IN); INTERNATIONAL AIDS VACCINE INITIATIVE, INC., New York, NY (US)

(72) Inventors: Jayanta BHATTACHARYA, Faridabad (IN); Nitin HINGANKAR, Faridabad (IN); Payel DAS, Faridabad (IN); Suprit DESHPANDE, Faridabad (IN); Pallavi KSHETRAPAL, Faridabad (IN); Ramachandran THIRUVENGADAM, Faridabad (IN); Amit AWASTHI, Faridabad (IN); Abbas Rizvi ZAIGHAM, Faridabad (IN); Devin SOK, New York, NY (US)

(21) Appl. No.: 18/686,251

(22) PCT Filed: Aug. 24, 2022
(86) PCT No.: PCT/IB2022/057923

§ 371 Date: Feb. 23, 2024

(30) Foreign Application Priority Data
Aug. 25, 2021 (IN) 202111038519

(51) Int. Cl. C07K 16/10; A61K 39/00; A61K 39/42; A61P 37/04

(52) U.S. Cl. CPC C07K 16/1003; A61K 39/42; A61P 37/04; A61K 2039/507; C07K 2317/76; C07K 2317/92

(57) ABSTRACT

The present invention relates to seven novel neutralizing human monoclonal antibodies (mAbs) THSC20.HVTR04, THSC20.HVTR06, THSC20.HVTR11, THSC20.HVTR26 THSC20.HVTR39, THSC20.HVTR55 and THSC20.HVTR88 and their nucleotide sequences isolated from a convalescent individual of Indian origin by antigen (RBD)-specific single B cell sorting and cloning of variable heavy and light IgG chain genes. The isolated mAbs demonstrate neutralization of wild type Wuhan strain and the following variants of concern: South African variant of concern (B.1.351), UK variant of concern (B.1.1.7), Brazilian variant of concern (PI), Delta (B.1.617.2) and Omicron (B.1.1.529) with exception of THSC20.HVTR39 unable to neutralize Gamma (P1). Of these THSC20.HVTR04 is able to potently neutralize Omicron BA.2 and BA.4/BA.5, THSC20.HVTR06 is able to neutralize Omicron BA.1, BA.2 and BA.5 with low potency, THSC20.HVTR11 potently neutralizes Omicron BA.1 and BA.2 and THSC20.HVTR26 neutralizes Omicron BA. 1 only with moderate potency. The present invention also discloses the binding affinity of the neutralizing mAbs to the receptor binding domain (RBD) representing Wuhan isolate (wild type). The present invention also, discloses the use of neutralizing monoclonal antibodies (mAbs) against SARS-CoV-2 for its diagnostic, prognostic, preventive and therapeutic purposes.

The present complete specification is a cognate application (under Section 10 & Section 9 (2) and Rule 13) from the Provisional Patent Applications 202111038519 & 202111059095 and the applicant is now hereby submitting the cognate application by continuing with Provisional Patent Application No. 202111038519.

### FIELD OF THE INVENTION

The present invention broadly pertains to the field of biotechnology. The present invention relates to discovery of human monoclonal antibodies capable of binding to and neutralizing human SARS-CoV-2 and its Variants of Concerns (VOC) for its diagnostic, prognostic, preventive and therapeutic purposes.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Globally severe acute respiratory syndrome coronavirus 2 (SARS-CoV-2) has infected over 500 million people with more than 6 million deaths (https://covid19.who.int/). The current COVID-19 pandemic highlights the need for broadly effective countermeasures to prevent or, at least, curtail future transmission and significantly reduce overall scope of the epidemic Although modern technologies have accelerated the development of vaccines for COVID-19, the ease and speed with which SARS-CoV-2 is spreading has highlighted an unmet need for therapies that are available before the infection spreads globally. Moreover, emergence of different variants of concern (VOC) in recent time has fueled rapid and increased transmission and associated with new variants. Importantly, with the second wave of COVID-19 in India and with emergence of unique variants such as B. 1.617 and its lineages B.1.617.1, B.1.617.2 and B.1.617.3, the health care system has been caught unaware and there is an urgent need for therapeutic intervention. Some monoclonal antibodies have been granted approval for clinical use such as Bamlanivimab as a monotherapy, and Bamlanivimab together with Etesevimab or Casirivimab with Imdevimab as a combination therapy (http://www.fdc.gov), but their utility in controlling COVID-19 is yet to be established and many of them have been found to be ineffective against the currently circulating Omicron variants (Takashita, E. et al. N Engl J Med. 2022 Aug. 4;387 (5): 468-470).

Several monoclonal antibodies have been isolated and characterized (http://epig.stats.ox.ax.uk/webapps/covbdub/) since the emergence of this pandemic from SARS-CoV-2 infected donors, however, most of these antibodies are still in research laboratories and have not yet found commercial significance. Notably, majority of these antibodies against the new emerging variants (such as Omicron BA.4/BA.5) are either very poorly effective or not effective and in some cases are yet to be tested.

Hence, there is an urgent need to discover and identify monoclonal antibodies that are effective against pan SARS-CoV-2 variants including the currently circulating SARS-CoV-2 variants like BA.2, BA.4, BA.5.

### Object of the Invention

An object of the invention is to provide potently novel human monoclonal antibodies against SARS-CoV-2 and their nucleotide sequences.

Another object of the present invention is to provide a process of obtaining these mAbs for effective prophylactic and therapeutic agents.

Another object of the invention is to provide a composition comprising the mAbs of the present invention.

Another object of the present invention is the utility of these mAbs in neutralising SARS-CoV-2 and its variants in diagnostics, prophylactics and therapeutics.

### BRIEF DESCRIPTION OF THE FIGURES

FIG. 1 depicts the identification of an Indian convalescent donor whose plasma demonstrated potent neutralization of replication competent SARS-CoV-2 in Vero-E6 cells. The virus stock was mixed with indicated dilution of donor plasma prior to adding to Vero-E6 cells. Post 72 hours of infection, cells were fixed and permeabilized with 1:1 cold methanol:acetone. The infectivity of the Vero-E6 was determined by immunostaining.

FIG. 2 depicts representative gating strategy for the antigen (receptor binding domain or RBD)-specific B cell sorting. Peripheral blood mononuclear cells (PBMCs) obtained from a convalescent donor C-03-0020 were stained with fluorophore conjugated antibodies to cell surface markers and streptavidin labelled RBD. Antigen (RBD)-specific single B cells were sorted using a FACS sorter. Singlet live CD19′C20+IgG⁺RBD⁺ cells were sorted into the 96 well plate prefilled with lysis buffer.

FIG. 3 depicts mapping of mutations in the variable region of the heavy and light chain IgG protein sequences. Variable regions of the heavy and light chain IgGs clones were sequenced and sequences were analyzed using the IMGT (International ImMunoGeneTics Information System, www.imgt.org) V-quest webserver tool. The mutated amino acid and their positions are highlighted.

FIG. 4 depicts expression, antigenicity and neutralization potential of the monoclonal antibodies. A. Supernatants harvested from HEK 293T cells co-transfected with variable heavy and light chains cloned into their respective IgG expression vectors were examined for expression of IgG by Fe-capture ELISA (black filled bar) and their ability to bind to SARS-CoV-2 receptor binding domain (RBD) used for B cell sorting by streptavidin ELISA (striped line bar). Non-specific IgG refers to IgG that showed efficient expression but did not bind to RBD. Non-functional IgG refers to IgG sequence that did not show any expression. B. Neutralization of pseudovirus expressing SARS-CoV-2 spike by cell supernatants expressing THS20.HVTR04 and THS20.HVTR26 produced in 293T cells. C. Expression of functional neutralizing IgGs was captured by SDS-PAGE.

FIG. 5 depicts binding affinity of THSC20.HVTR04, THSC20.HVTR06, THSC20.HVTR11, THSC20.HVTR26, THSC20.HVTR39, THSC20.HVTR55 and THSC20.HVTR88 to SARS-CoV-2 wild type receptor binding domain (RBD) by biolayer interferometry in BLI-Octet platform. Biotinylated wild type SARS-CoV-2 RBD antigen was immobilized on Streptavidin (SA) biosensors and binding affinity of monoclonal antibodies (mAbs) to RBD was tested using threefold serial dilutions of mAbs starting with 33.3 nM to lowest 0.41 nM (five different concentrations were tested). Association and dissociation were assessed for 500 seconds each. Data shown is reference-subtracted and aligned using Octet Data Analysis software v10.0.1.6 (Forte Bio). Curve fitting was performed using a 1:1 binding model and Kon, Koff and KD values were determined with a global fit.

FIG. 6 depicts Binding of mAbs to SARS-CoV-2 spike protein expressed on 293T cells as assessed by mean fluorescent intensity (MFI) in a flow cytometry.

FIG. 7. A. depicts neutralization of SARS-CoV-2 pseudoviruses expressing wild type (Wuhan), and other variants of concern; VOC (Alpha, Beta, Gamma, Delta) and variants of interest; VOIs (Kappa, Delta Plus) spike sequences by THSC20.HVTR04 and THSC20.HVTR26 mAbs. Other known mAbs (REGN10933, REGN10987, CC12.1 and CC6.36) were included in the experiment as benchmarking controls for head to head comparison. B. Neutralization breadth of THSC20.HVTR11 & THSC20.HVTR55 mAbs. C. Comparison of neutralization breadth and potency of all the seven mAbs against SARS-CoV-2 variants. Values indicate dose of mAb (μg/mL) that showed 50% reduction in infectivity (expressed as IC50 values) in pseudovirus neutralization assay.

FIG. 8. Comparison of neutralizing breadth and potency of all the mAbs isolated from the donor C-03-0020 in pseudovirus neutralization assay. Representative dose response curves from experiment with each concentration response tested in duplicate. THSC20HVTR04 and THSC20.HVTR26 mAbs were found to show maximum neutralization potency (lower panel, right) as determined by their IC50 values, obtained by non-linear regression four parameter curve fit method in GraphPad Prism. Shown values are mean with SEM.

FIG. 9. Live authentic virus foci reduction neutralization test (FRNT). A. Neutralization of SARS-CoV-2 variants (Wuhan, Alpha, Beta, Kappa and Delta) by the two top potent mAbs (THSC20.HVTR04 and THSC20.HVTR26) in Vero-E6 cells. B. Neutralization of replication competent live Omicron BA.1. BA.2 and BA.5 by THSC20.HVTR04, THSC20.HVTR06, THSC20.HVTR11 and THSC20.HVTR26.

FIG. 10. depicts epitope binning of mAbs performed using BLI-Octet platform. Biotinylated SARS-CoV-2 RBD was captured using streptavidin biosensor and indicated mAbs at a concentration of 100 ng/ml first incubated for 10 min followed by incubation with 25 μg/ml of competing antibodies for 5 min. The epitope specificity of the mAbs were compared with few published neutralizing mAbs (Rogers, T. F. et al. 2020, Science 21;369 (6506): 956-963).

FIG. 11. depicts ACE2-mAb competition for RBD. Inhibition of SARS-CoV-2 RBD binding by five mAbs to the cell surface hACE2 was assessed by flow cytometry.

FIG. 12. depicts prophylactic efficacy of THSTC20.HVTR04 and THSC20.HVTR26 mAbs in human ACE-2 transgenic K18 mouse model challenged with SARS-CoV-2 (Wuhan strain). K18 ACE-2 mice were divided into following groups: untreated, pre-treated with non-SARS-CoV-2-specific IgG control, pre-treated with THSC20.HVTR04, pre-treated with THSC20.HVTR26 and pre-treated with combination of THSC20.HVTR04 and THSC20.HVTR26. A. Changes in body weight post infusion of novel COVIDS mAbs in the infection course; (B), percent reduction in body weight at day 5 post virus challenge in mAb treated and untreated groups; C. Lung viral load at day 6 in mAb treated and untreated groups and D. correlation of virus load and percent change in body weight at day 6.

FIG. 13. illustrates the prophylactic effect of THSC20.HVTR04 and THSC20.HVTR26 in combination against SARS-CoV-2 Delta variant on preventing body weight loss at four different concentrations as 10 mg, 2.5 mg, 0.625 mg and 0.156 mg mAb per kg body weight, day-wise and on day 6, respectively (Upper panel), Lung viral load assessed at day 6 and (Left middle panel) serum IgG titer in mice sera detected at day 0 at the time of virus challenge (Right middle panel), correlation of percent body weight change with lung viral load at day 6 and (Left lower panel) correlation of percent body weight change with serum IgG titer at day 0 at the time of virus challenge in groups of different mAbs doses (Right lower panel).

FIG. 14. illustrates poly-reactivity assessment of THSC20.HVTR04 and THSC20.HVTR26 mAbs using CHO soluble membrane protein (SMP) by ELISA. Three-fold serial dilutions of mAbs starting with 100 ug/mL were tested.

### SUMMARY OF THE INVENTION

The present application relates to novel human monoclonal antibodies (mAbs) THSC20.HVTR04, THSC20.HVTR06, THSC20.HVTR11, THSC20.HVTR26, THSC20.HVTR39, THSC20.HVTR55 and THSC20.HVTR88 and their nucleotide sequences isolated from a convalescent and unvaccinated individual of Indian origin that targets RBD of the viral spike protein. The two most potently neutralizing mAbs (THSC20.HVTR04 and THSC20.HVTR26) demonstrate neutralization of wild type Wuhan strain, South African variant of concern (B.1.351 or Beta), UK variant of concern (B.1.1.7 or Alpha), Delta variant of concern (B.1.617.2), Gamma variant of concern (PI), Kappa variant of interest (B.1.617.1) and Delta Plus variant of interest. In addition, few of these mAbs (such as THSC20.HVTR04, THSC20.HVTR06, THSC20.HVTR11, THSC20.HVTR26) also demonstrated neutralization of Omicron variants. In particular, THSC20.HVTR06, THSC20. HVTR11 and THSC20.HVTR26 neutralizes Omicron BA.1 variant; THSC20.HVTR04, THSC20.HVTR06 and THSC20.HVTR11 neutralizes Omicron BA.2 also and THSC20.HVTR04 neutralizes Omicron BA.4 and BA.5. The present invention also discloses the binding affinity of all the neutralizing mAbs to the RBD representing Wuhan isolate (wild type). The present invention also, discloses the use of neutralizing monoclonal antibodies (mAbs) against SARS-CoV-2 for its diagnostic, prognostic, preventive and therapeutic purposes.

In an embodiment, the invention provides a composition of monoclonal antibodies for the treatment of SARS-CoV-2 virus infection wherein the monoclonal antibodies exhibit strong binding to receptor binding domain of the viral spike protein of SARS-CoV-2, comprises:
a) THSC20.HVTR04 comprising variable Heavy chain IgG sequence of SEQ ID NO. 1 and variable light chain IgG sequence of SEQ ID NO. 2
b) THSC20.HVTR06 comprising variable heavy chain IgG sequence of SEQ ID NO. 3 and variable light chain IgG sequence of SEQ ID NO. 4
c) THSC20.HVTR11 comprising variable Heavy chain IgG sequence of SEQ ID NO. 5 and variable light chain IgG sequence of SEQ ID NO. 6
d) THSC20.HVTR26 comprising variable heavy chain IgG sequence of SEQ ID NO. 7 and variable light chain IgG sequence of SEQ ID NO. 8
e) THSC20.HVTR39 comprising variable heavy chain IgG sequence of SEQ ID NO. 9 and variable light chain IgG sequence of SEQ ID NO. 10
f) THSC20.HVTR55 comprising variable Heavy chain IgG sequence of SEQ ID NO. 11 and variable light chain IgG sequence of SEQ ID NO. 12
g) THSC20.HVTR88 comprising variable heavy chain IgG sequence of SEQ ID NO. 13 and variable light chain Ig sequence of SEQ ID NO. 14

In an embodiment, the composition of monoclonal antibodies comprises at least THSC20.HVTR04 and THSC20.HVTR26.

In an embodiment, the composition of monoclonal antibodies comprises at least THSC20.HVTR06, THSC20.HVTR11, THSC20.HVTR39, THSC20.HVTR55 and THSC20.HVTR88.

In an embodiment, there is provided a therapeutic composition comprising combination of THSC20.HVTR04 and THSC20.HVTR26 mAbs against SARS-CoV-2 Delta variant wherein the therapeutic composition is present in an amount of 0.625 mg/kg body weight.

In an embodiment, there is provided a method for isolating monoclonal antibodies (mAbs) against SARS-CoV-2 as described above, comprising the steps of:
a) selection of a donor with high neutralization titer of antibodies in the plasma;
b) Sorting of SARS-CoV-2 specific single IgG positive B cells using biotinylated SARS-CoV-2 RBD protein as an antigen, which were subsequently used as source for amplification of heavy and light chain variable genes of IgG from single B cell
c) Novel broadly neutralizing antibodies (bnAbs) were obtained by emphasizing neutralization as the initial screen.

In an embodiment, there is provided a method for obtaining the monoclonal antibodies (mAbs) as described above, comprising the steps of:
i) Sorting of SARS-CoV-2 specific single B cells from a donor PBMC sample for neutralization activity against a plurality of SARS-CoV-2 variants
ii) RT-PCR and amplification of variable heavy and light IgG sequences
iii) Cloning of heavy and light chain variable functional antibody genes from a single B cell that exhibits neutralization activity tested by pseudovirus and live virus neutralization assays
iv) Selection of the desired mAb clones and scaling up for IgG purification by co-transfection of plasmid DNA expressing variable heavy and light chain IgG sequences in Expi-293 cells

### DETAILED DESCRIPTION OF THE INVENTION

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in this application are those well-known and commonly used in the art.

The present invention is drawn to human monoclonal antibodies that are novel based on their sequences and that exhibit strong binding to Wuhan RBD of which two (THSC20.HVTR04 and THSC20.HVTR26) demonstrated potent neutralization of pseudoviruses expressing spike protein of SARS-CoV-2 (Wuhan), Alpha (B.1.1.7), Beta (B.1.351), Gamma (PI), Delta (B.1.617.2), Kappa (B.1.617.2) and Delta plus spikes. These two monoclonal antibodies (THSC20.HVTR04 and THSC20.HVTR26) were also found to potently neutralize live SARS-CoV-2 primary isolates (Alpha, Beta, Delta, Kappa). THSC20.HVTR04 was also found to neutralize currently circulating Omicron BA.2 and BA.4/BA.5 variants, while THSC20.HVTR26 neutralizes Omicron BA.1 variant. The neutralizing monoclonal antibodies of the present invention would be useful for therapeutic and prophylaxis purpose against SARS-CoV-2 infection with variants of concern, including wild type Wuhan strain, South African variant of concern (B.1.351) and UK variant of concern (B.1.1.7), Delta (B.1.617.2) and Omicron (B.1.1.529). Other five monoclonal antibodies (THSC20.HVTR06, THSC20.HVTR011, THSC20.HVTR26, THSC20.HVTR39, THSC20.HVTR55 and THSC20.HVTR88) binds strongly to the SARS CoV-2 RBD in addition to neutralization activity against SARS COV-2 variants of concern.

The present invention also discloses the binding affinity of the neutralizing mAbs to the RBD protein representing Wuhan isolate (wild type).

The novel monoclonal antibodies are represented by their Sequence IDs as below;
THSC20.HVTR04 (Variable Heavy chain IgG sequence)—SEQ ID NO. 1
THSC20.HVTR04 (Variable light chain IgG sequence)—SEQ ID NO. 2
THSC20.HVTR06 (Variable heavy chain IgG sequence)—SEQ ID NO. 3
THSC20.HVTR06 (Variable light chain IgG sequence)—SEQ ID NO. 4
THSC20.HVTR11 (Variable heavy chain IgG sequence)—SEQ ID NO. 5
THSC20.HVTR11 (Variable light chain IgG sequence)—SEQ ID NO. 6
THSC20.HVTR26 (Variable heavy chain Ig (sequence)—SEQ ID NO. 7
THSC20.HVTR26 (Variable light chain IgG sequence)—SEQ ID NO. 8
THSC20.HVTR39 (Variable heavy chain IgG sequence)—SEQ ID NO. 9
THSC20.HVTR39 (Variable light chain IgG sequence)—SEQ ID NO. 10.
THSC20.HVTR55 (Variable heavy chain IgG sequence)—SEQ ID NO. 11
THSC20.HVTR55 (Variable light chain IgG sequence)—SEQ ID NO. 12
THSC20.HVTR88 (Variable heavy chain IgG sequence)—SEQ ID NO. 13
THSC20.HVTR88 (Variable light chain IgG sequence)—SEQ ID NO. 14

The monoclonal antibodies (mAbs) of the present invention were obtained by antigen (Wuhan RBD)-specific B cell sorting and cloning technique, targets epitopes on RBD. The monoclonal antibodies (THSC20.HVTR04 and THSC20.HVTR26) of the present invention were found to be most potent neutralizing human monoclonal antibodies and are capable of potently neutralizing wild type SARS-CoV-2 (Wuhan), Alpha, Beta, Gamma, Delta, Delta Plus and Kappa.

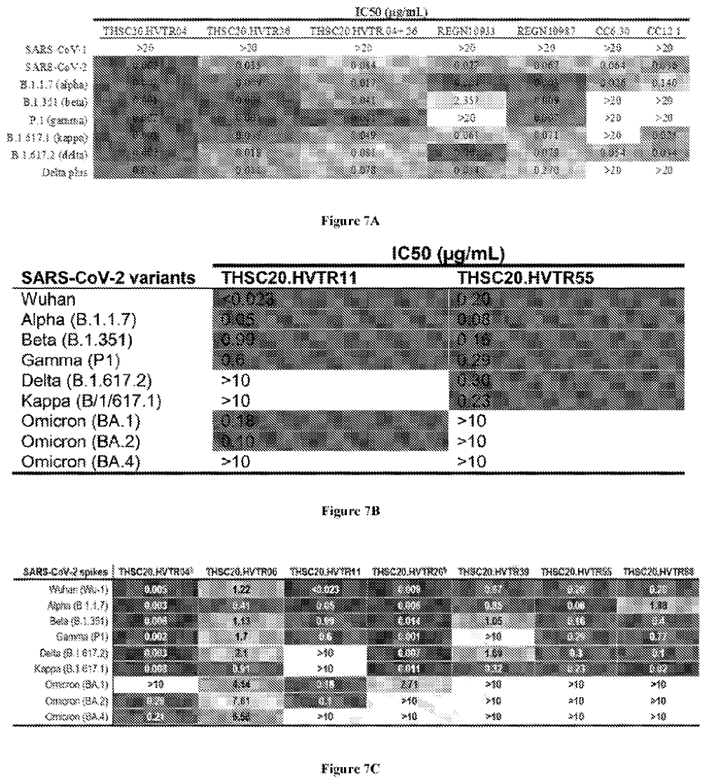

The monoclonal antibodies (mAbs) of the present invention when compared with some known neutralising mAbs (See FIG. 7) were found to demonstrate distinct epitope specificities with most of them through epitope binding experiment.

The various characteristic parameters of the monoclonal antibodies (mAbs) of the present invention are set out Table 1:

TABLE 1
Characteristics of the monoclonal antibodies(mAbs) of the present invention
Heavy chain IgG

| | | % identity | | % identity | | |
|---|---|---|---|---|---|---|
| | | to germline | | to germline | | |
| | V gene & | (V region) | J gene & | (J region) | | CDHR3 |
| mAb ID | allele | (nucleotide) | allele | (nucleotide) | CDHR3 sequence | length |
| THSC20. | IGHV3-30- | 96.88% | IGHJ2*01 | 90.57% | CASVADTAMVPEWYF | 16 |
| HVTR04 | 3*01 F | | F | | DLW | |
| THSC20. | IGHV7-4- | 96.53% | IGHJ6*02 | 96.77% | CARVLGDQQVYGGDD | 23 |
| HVTR06 | 1*02 F | | F | | YYYYYAMDVW | |
| THSC20. | IGHV1-69*02 | 97.57% | IGHJ4*02 | 100% | CARIRGYSGYGSSYYF | 17 |
| HVTR11 | F | | F | | DYW | |
| THSC20. | IGHV3-53*04 | 98.25% | IGHJ3*02 | 96% | CARLVGALTNIVVSGD | 21 |
| HVTR26 | F | | F | | GGAFDIW | |
| THSC20. | IGHV4-39*07 | 94.50% | IGHJ3*02 | 98% | CARNIFYYDRSGSSH | 20 |
| HVTR39 | F | | F | | HDAFDIW | |
| THSC20. | IGHV5-51*01 | 96.88% | IGHJ4*02 | 89.58% | CARRDRWELDPLDYW | 13 |
| HVTR55 | F | | F | | | |
| THSC20. | IGHV1-18*01 | 98.61% | IGHJ4*02 | 95.83% | CAREGKYSSGWSYSE | 18 |
| HVTR88 | F | | F | | YFDYW | |

Light chain IG

| | | % identity | | % identity | | |
|---|---|---|---|---|---|---|
| | | to germline | | to germline | | |
| | V gene | (V region) | J gene & | (J region) | | CDHR3 |
| mAb ID | & allele | (nucleotide) | allele | (nucleotide) | CDR3 sequence | length |
| THSC20.HVTR04 | IGLV2- | 97.92% | IGLJ3*02 | 97.37% | CCSYAGSWVF | 8 |
| | 11*01 P | | F | | | |
| THSC20.HVTR06 | IGLV3- | 97.49% | IGLJ3*02 | 97.74% | CQAWDNSGWVF | 8 |
| | 1*01 F | | | | | |
| THSC20.HVTR11 | IGLV1- | 99.65 | IGLJ2*01F | 94.74% | CQSYDSSLSHEVF | 11 |
| | 40*01F | | or | | | |
| | | | IGLJ3*01F | | | |
| THSC20.HVTR26 | IGLV2- | 97.57% | IGLJ3*02 | 97.22% | CCSYAGSSTWVF | 10 |
| | 23*02 F | | F | | | |
| THSC20.HVTR39 | IGLV3- | 93.01% | IGLJ3*02 | 100% | CQVWDRGSDVWVF | 11 |
| | 21*02 F | | F | | | |
| THSC20.HVTR55 | IGKV1- | 97.85% | IGKJ2*01 | 94.87% | CQHYNGYPYTF | 9 |
| | 5*03 F | | F | | | |
| THSC20.HVTR88 | IGKV3- | 100% | IGKJ1*01 | 92.11% | CQQYGSSLWTF | 9 |
| | 20*01 F | | F | | | |

The present invention discloses a novel monoclonal antibody (mAbs) against SARS-CoV-2. The novel mAb THSC20.HVTR04, as disclosed herein nucleotide sequences having heavy chain and light chair of unique SEQUENCE ID NO. 1 AND SEQ ID NO. 2 coding the variable heavy and light chain IgG regions. The novel mAb THSC20.HVTR26, as disclosed herein nucleotide sequences having heavy chain and light chain of unique SEQUENCE ID NO. 7 AND SEQ ID NO. 8 coding the variable heavy and light chain IgG regions.

The invention relates to potent, neutralizing monoclonal antibodies (mAbs) wherein the antibody neutralizes one or more variants of SARS-CoV-2, which may be selected from representing Wuhan isolate (wild type), Alpha (B.1.1.7; VOC), Beta (B.1.351; VOC), Gamma (PI), Delta (B.1.6517.2), Omicron (B.1.1.529) and its variants (BA.1, BA.2, BA.4/BA.5) and have an IC50 value of less than 0.5 μg/ml.

The invention also provides a method for obtaining monoclonal antibodies (mAbs) comprising the steps of:
i. sorting of SARS-CoV-2 specific single B cells from a donor PBMC sample for neutralization activity against a plurality of SARS-CoV-2 variants,
ii. RT-PCR and amplification of variable heavy and light IgG sequences
iii. cloning of heavy and light chain variable functional antibody genes from a single B cell that exhibits neutralization activity tested by pseudovirus neutralization assay.
iv. Selection of the desired mAb clones and scaling up for IgG purification by co-transfection of plasmid DNA expressing variable heavy and light chain IgG sequences in Expi-293 cells.
v. Purification of the IgG using Protein A/G column.

The monoclonal antibodies (mAbs) of the present invention were obtained from Peripheral Blood Mononuclear Cells (PBMCs) or B cells. The PBMCs were isolated from an individual, who recovered from SARS-CoV-2 infection selected for SARS-CoV-2 neutralizing activity in the plasma. Antibodies generated from single B cells were subjected to a primary screen of neutralization assay using pseudovirus to determine neutralization potential and the binding reactivity to RBD was determined by ELISA.

Any suitable host cell/vector system may be used for expression of the DNA sequences encoding the antibody molecules of the present invention or fragments thereof. Eukaryotic, e.g. mammalian, host cell expression systems may be used for production of larger antibody molecules, including complete antibody molecules. Suitable mammalian host cells include CHO, HEK 293T, HEK 293F, Expi-293.

The present invention also provides a process to produce an antibody protein that comprises of transfection of two plasmids (one encoding variable heavy IgG chain sequence and another encoding variable light IgG sequence) into mammalian cell line (e.g., Expi293) under conditions suitable for leading to expression of protein from DNA encoding the antibody molecule of the present invention and isolating the antibody molecule.

The antibody molecule may comprise of only a heavy or light chain variable region polypeptide. in which case only a heavy chain or light chain variable region polypeptide coding sequence needs to be used to transfect the host cells. For production of products comprising both heavy and light chains, the cell line may be transfected with two vectors, a first vector encoding a light chain variable region polypeptide and a second vector encoding a heavy chain variable region polypeptide. Alternatively, a single vector may be used, the vector including sequences encoding light chain and heavy chain polypeptides.

The antibodies of the present invention may be produced by a process comprising of the following steps:
i) Co-transfection of plasmid DNA encoding variable heavy IgG chain sequence and plasmid DNA encoding variable light IgG chain sequence into a mammalian cell line (e.g., Expi293 or ExpiCHO),
ii) Expressing nucleic acid sequences of variable heavy and light chain IgG in mammalian cell lines (e.g., Expi293 or ExpiCHO) as producer cells,
iii) Harvesting cell supernatant between 4-5 days.
iv) Purifying the antibody as IgG using Protein A/G beads using a column.

In yet another embodiment, the present invention envisages compositions comprising the monoclonal antibodies (mAbs) of the present invention, including a polypeptide, antibody, or modulator of the present invention, at a desired degree of purity, and a pharmaceutically acceptable carrier, excipient, or stabilizer. Compositions may also be done to enhance the stability of the polypeptide or antibody during storage, e.g., in the form of lyophilized compositions or aqueous solutions.

The composition may also contain one or more additional therapeutic agents suitable for the treatment of the particular indication, e.g., infection being treated, or to prevent undesired side-effects. Preferably, the additional therapeutic agent has an activity complementary to the polypeptide or antibody of the present invention, and the two do not adversely affect each other. For example, in addition to the polypeptide or antibody of the invention, an additional or second antibody, anti-viral agent, and/or anti-infective agent may be added to the composition. Such molecules are suitably present in the pharmaceutical composition in amounts that are effective for the purpose intended.

In another embodiment, the mAb of the present invention has diagnostic, pharmaceutical, immunogenic, immunotherapy, immunological applications. It may also be used to design vaccines.

These antibodies can be used as prophylactic or therapeutic agents upon appropriate composition, or as a diagnostic tool.

In another embodiment, the mAb of the present invention may be administered in a dose of 0.1 mg/Kg body weight to 100 mg/Kg body to elicit protective and therapeutic responses.

The expression vectors carrying the antibody heavy and light chain variable region genes can be used in various ways e.g. as a source of monoclonal antibodies, as a source of nucleic acid (DNA or mRNA) encoding a monoclonal antibody of interest, for research, etc.

Monoclonal and recombinant antibodies are particularly useful in identification and purification of the individual polypeptides or other antigens against which they are directed. The antibodies of the invention have additional utility in that they may be employed as reagents in immunoassays, radioimmunoassays (RIA) or enzyme-linked immunosorbent assays (ELISA). In these applications, the antibodies can be labelled with an analytically detectable reagent such as a radioisotope, a fluorescent molecule or an enzyme. The antibodies may also be used for the molecular identification and characterization (epitope mapping) of antigens.

The mAbs of the present invention may comprise hitherto unknown/unique may be used for future immunogen design; for the development of an immunoassay kit for the detection of SARS-CoV-2 specific antigen in a sample of body fluid. Therefore, this invention may have commercial, therapeutic, diagnostic, immunologic value in near to distant future.

The following examples are included to demonstrate preferred embodiments of the invention. It should be appreciated by those of skill in the art that the techniques disclosed in the examples which follow represent techniques used/followed by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Isolation of SARS-CoV-2 RBD-Specific Monoclonal Antibodies (mAbs) from a Convalescent Individual

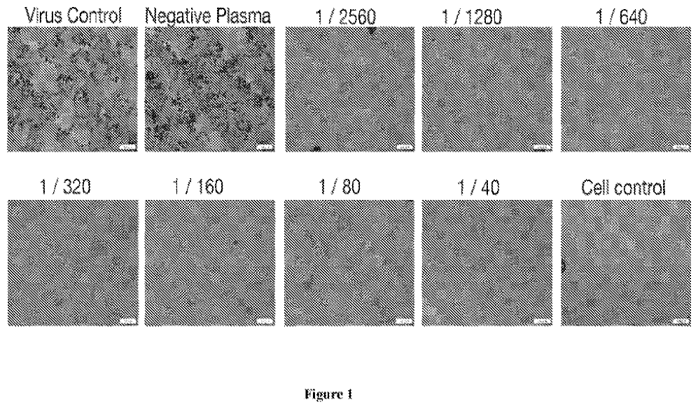

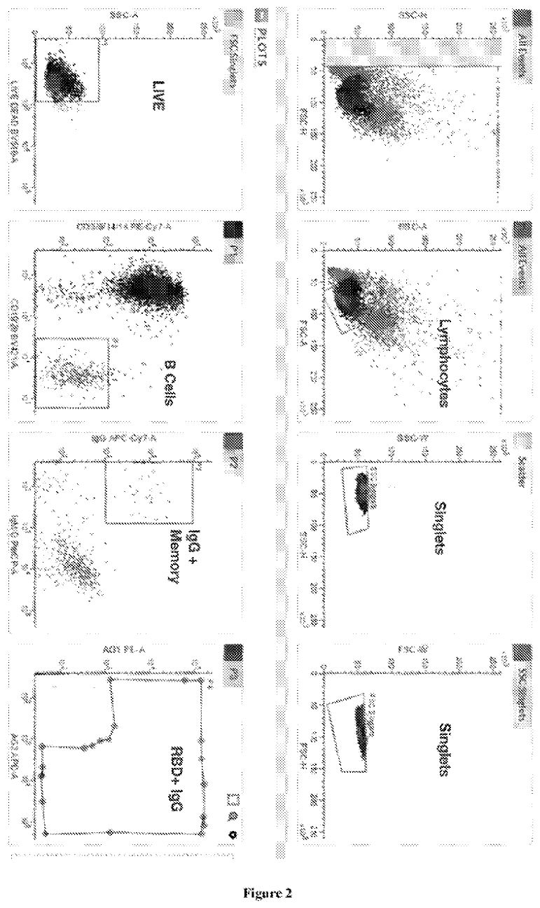

The present invention identifies an Indian individual (donor ID: C-03-0020) recovered from SARS-CoV-2 infection whose plasma was found to demonstrate potent neutralization of SARS-CoV-2 (FIG. 1). The infectivity assay was done in Vero-E6 cells. Serial 2-fold diluted plasma (heat-inactivated) prepared from the donor C-03-0020 blood sample was mixed with virus and incubated for 1 hour at 37° C. and subsequently added to Vero-E6 cells in 96-well tissue culture plate. The plate was kept in a CO₂ incubator under humidified condition. Post 48-hour incubation, cells were fixed with cold acetone:methanol (1:1) and infected cells were detected by immortalizing using pooled SARS-CoV-2 convalescent plasma as described in J Virol, 2004, 78:6915-26. Antigen-specific memory B cell sorting was performed using BD FACS Melody sorter. Fluorescent-labelled antibodies to cell surface markers were used. Avi-tagged RBD protein subsequently labelled with biotin was coupled to streptavidin-PE and streptavidin-APC by incubating at 4° C. for 1 hour at 4:1 molar ratio to prepare probes. Cryo-preserved PBMCs were thawed at 37° C. in water bath and washed with RPMI medium containing 10% fetal bovine serum (FBS). Cells were first labelled with streptavidin conjugated RBD probes (200 nM final) for 30 min and then with antibodies for surface markers (CD3: PE-Cy7; CD8: PE-Cy7; CD14: PE-Cy7; CD16: PE-Cy7; CD19: BV421; CD20: BV421; IgD: PerCP-Cy5.5; IgG: APC-H7 for 20 min in FACS buffer (PBS 1% FBS, 1.0 mM EDTA) on ice. Live/Dead fixable aqua blue cell Stain was used to stain the cells for another 10 minutes on ice as per the manufacturer's instructions. Cells were washed with FACS buffer and filtered with 70-μm cell mesh (BD Biosciences). Single antigen-specific (RBD+) memory B cells (CD3⁻CD8⁻CD14⁻CD16⁻CD19⁺CD20⁺IgD⁻IgG⁺) were sorted into individual wells of a 96-well plate prefilled with 20 ul of lysis buffer containing reverse transcriptase (RT) Buffer, IGEPAL, DTT and RNAseOUT using a BD FACS Melody sorter at 5° C. Plates were sealed, snap-frozen on dry ice and stored at-80° C. until used. (FIG. 2)

### Example 2: Amplification and Cloning of Variable Heavy and Light IgG Chains

Superscript III Reverse Transcription kit was used to prepare cDNA from sorted cells, cDNA master mix containing dNTPs, random hexamers, IgG gene-specific primers and RT enzyme was added to generate cDNA. Heavy and light-chain variable regions of IgG were amplified in independent nested PCR reactions using specific primers. First round PCR amplification was performed using HotStar Taq DNA Polymerase and second round nested PCR was performed using Phusion HF DNA polymerase. Specific restriction enzyme cutting sites (heavy chain, 5′-AgeI/3′-SalI; kappa chain, 5′-AgeI/3′-BsiWI; and lambda chain, 5′-AgeI/3′-XhoI) were introduced in the second round PCR primers in order to clone into the respective expression vectors. Amplified PCR products were verified on the agarose gel and wells with double positives (with amplification of both Heavy and Light chain variable region from the same well) were identified and selected for subsequent cloning experiments. PCR products were digested with specific restriction enzymes, purified and cloned in-frame into expression vectors using the Quick Ligase cloning system according to the manufacturer instructions. Ligation reactions were transformed into NEB 5-alpha competent E. coli cells. plated on LB agar plates containing ampicillin and incubated overnight at 37° C. in incubator. Colonies with desired inserts were screened by colony PCR and used for preparation of plasmid DNA. Plasmid DNA with insert in correct orientation were further confirmed by restriction digestion.

### Example 3: Screening for Functional Monoclonal Antibodies for Selection of Ones Capable of Virus Neutralization

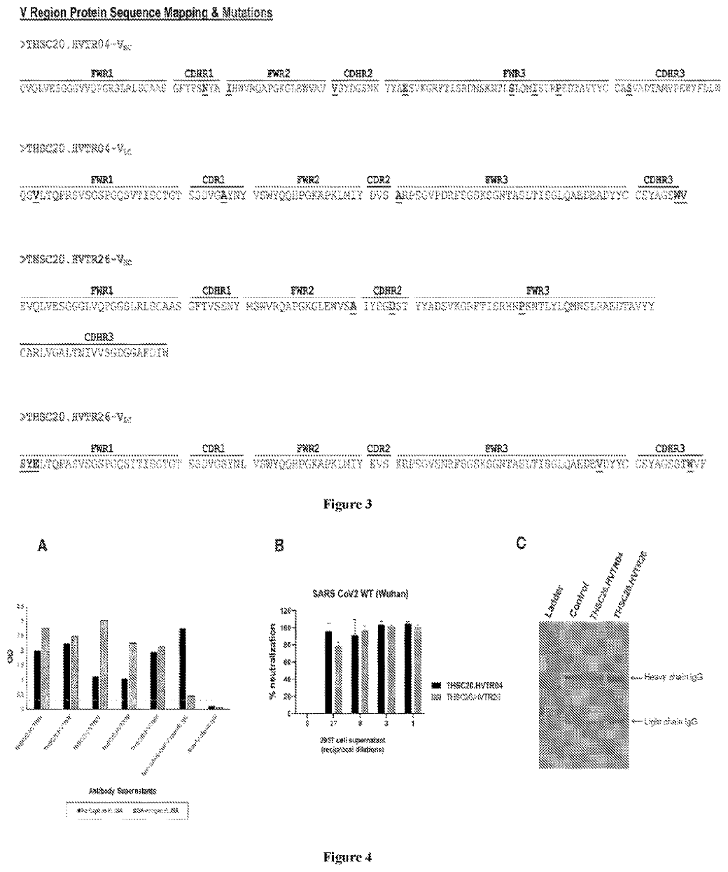

Plasmid DNA containing variable heavy and light IgG chain sequences were co-transfected in HEK 293T cells (ATCC) using Fugene transfection reagent in 24 well plates for preparing antibody supernatant for initial screening for their expression and antigen specificity as detailed in the following section. Sanger sequencing were carried out to obtain the nucleotide and amino acid sequences of variable heavy and light IgG chains. Analysis of mAb sequences were carried out using the IMGT (www.imgt.org) V-quest webserver tool. (FIG. 3).

The mAb clones were first assessed for their ability to express by capture ELISA for the detection of IgG expression. For this, MaxiSorp high protein binding 96 well ELISA plate was coated with 2 μg/mL goat anti-human Fc antibody and incubated for overnight at 4° C. Next day after washing, plates were blocked with 3% BSA in PBS (pH 7.4) for 1 hour at room temperature. After 3 times of washing with 1×PBS containing 0.05% tween 20 (PBST), the cell supernatants harvested post transfection of antibody constructs in HEK 293T cells were added and incubated for 1 hour at room temperature. This was followed by addition of alkaline phosphatase-conjugated goat anti-human F(ab′)2 antibody at 1:1000 dilution in 1% bovine serum albumin (BSA) incubated for an hour at room temperature. After the final wash, phosphatase substrate was added into the wells and absorption was measured at 405 nm on a 96 well microtiter plate reader. (FIG. 4A).

The functional mAbs were next assessed for their ability to bind to SARS-CoV-2 RBD monomeric protein by ELISA. For this, 2 μg/mL of Streptavidin was coated onto each wells of Nunc MaxiSorp high protein-binding 96 well ELISA plate and incubated overnight at 4° C. Next day after washing, plates were blocked with 3% BSA in PBS (pH 7.4) for 1 hour at room temperature. 2 μg/mL of Biotinylated-RBD protein was subsequently added and incubated the plate for 2 hours at room temperature. After washing the plates for 3 times with PBST, cell supernatants at various dilutions were added to the wells and the plate was further incubated for 1 hour at room temperature. Finally, HRP (horse radish peroxidase) conjugated anti-human IgG Fe secondary antibody was added at a dilution of 1:1000 containing 1% BSA and the plate was incubated for an hour at room temperature. After the final wash, TMB substrate was added and subsequently 1N H₂SO₄ was added to stop the reaction. The absorption was measured at 450 nm. (FIG. 4A). Neutralization activity of the supernatants harvested from the transfection of the heavy and light chain plasmids for two mAbs were screened against SARS-CoV-2 pseudovirus with three-fold dilution series of the supernatant. (FIG. 4B)

### Example 4: Preparation and Purification of IgG

The IgGs representing the mAbs were produced in either HEK 293T or Expi293 cells. Plasmid DNA expressing variable heavy and light IgG chains were transiently transfected into HEK293T or Expi293 cells using polyethylenimine (PEI). After 4-5 days of incubation, supernatants were harvested by centrifugation and filtered through 0.2 μm membrane filter. Supernatants were then flowed slowly on to the Protein A beads in the column at 4° C. in order to capture the secreted antibodies. Beads in the column were washed with five column volumes of 1×PBS at room temperature. Antibodies were eluted in two to three column volumes of 100 mM Glycine (pH 2.5) and immediately neutralized with IM Tris-HCL (pH 8.0). Eluted antibodies were dialyzed using 10K MWCO SnakeSkin dialysis tubings against 1×PBS thrice and then concentrated in 30 kDa MWCO Amicon Ultra-15 Centrifugal Filter Units. Antibody solutions were finally filtered through 0.2 am syringe filter before used for the further experiments. Concentration of IgG was measured in a NanoDrop spectrophotometer and IgG heavy and light chain bands were visualized in a 12% SDS PAGE. (FIG. 4C)

### Example 5: Assessment of Binding Kinetics of mAbs to SARS-CoV-2 RBD by Biolayer Interferometry

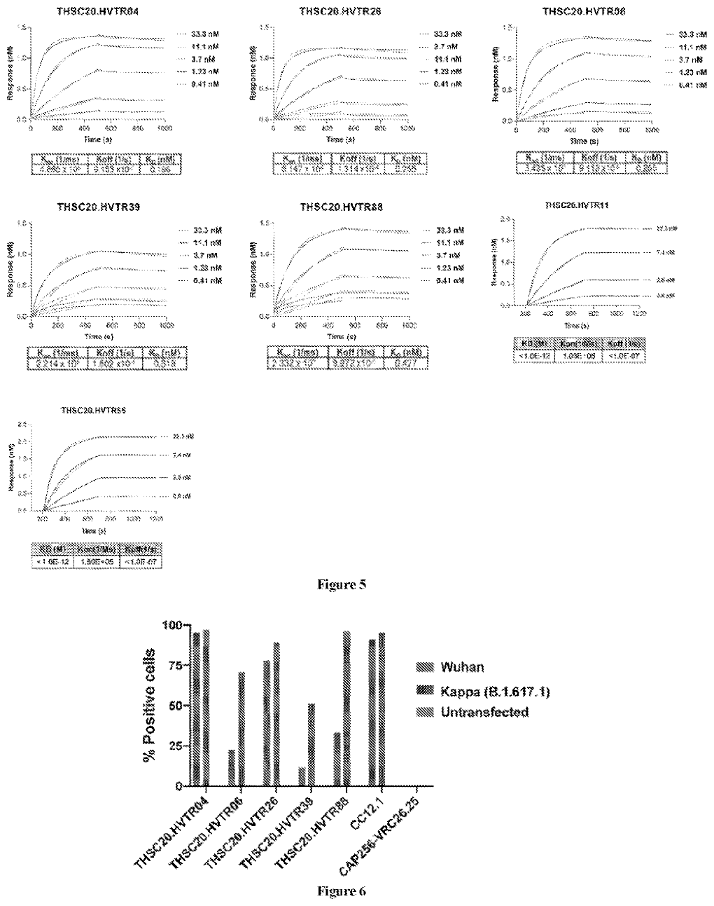

Streptavidin (SA) biosensors were used to assess the binding kinetics of mAbs with SARS-CoV-2 RBD in PBST (PBS containing 0.02% Tween 20) at room temperature (around 25° C.) and 1,000 r.p.m. shaking on an Octet RED instrument. Sensors were first soaked in PBS for 15 minutes before being used to capture biotinylated SARS-CoV-2 RBD protein. RBD was loaded to the biosensors up to a level of 1.0-1.2 nm. Biosensors were then immersed into PBS for 100 seconds and then immersed into wells containing specific concentration of a mAb dissolved in PBST (PBS containing 0.02% Tween 20) for 500 seconds to measure association. A three folds dilution series with five different concentrations (33.3, 11.1, 3.7, 1.23, and 0.41 nM) was prepared for each mAb. Biosensors were next dipped into wells containing PBST′ for 500 seconds to measure dissociation. Data were reference-subtracted and aligned to each other using Octet Data Analysis software v10.0.1.6 based on a baseline measurement. Curve fitting was performed using a 1:1 binding model and data for all the five concentrations of mAbs. Kon, Koff and KD values were determined with a global fit. As shown in FIG. 5, THSC20.HVTR04 and THSC20.HVTR26 were found to strongly bind to SARS-CoV-2 receptor binding domain (RBD) antigen with K_{D} of 0.19 nM and 0.22 nM respectively. (FIG. 5)

### Example 6: Cell Surface Spike Binding Assay

Since we used monomeric RBD to isolate the mAbs, we further assessed their ability bind to trimeric spike protein expressed on the cell surface. The binding of mAbs to the SARS-CoV-2 spikes expressed on the HEK 293T cell-surface was assessed as described previously with some modifications [20]. Briefly, HEK293T cells were transfected with the three plasmids used to generate SARS-CoV-2 pseudovirus (SARS-CoV-2 MLV-gag/pol, MLV-CMV-luciferase and SARS-CoV-2 spike plasmids). After incubation for 36-48 h at 37° C., cells were trypsinized and a single cell suspension was prepared which was distributed into 96-well U bottom plates. 3-fold serial dilutions of mAbs starting at 10 ng/ml and up to 0.041 μg/mL were prepared in 50 μl/well and added to the spike expressing as well as un-transfected 293T cells for 1 hour on ice. Cells were subsequently washed twice with FACS buffer (1×PBS, 2% FBS, 1 mM EDTA) and then stained with 50 μl/well of 1:200 dilution of R-Phycoerythrin AffiniPure F(ab′)₂ Fragment Goat Anti-Human IgG, F(ab′)₂ fragment specific antibody (Jackson ImmunoResearch Inc.) for 45 min. Cells were finally stained firstly with 1 LIVE/DEAD fixable aqua dead cell stain (ThermoFisher) in the same buffer for another 15 minutes and subsequently washed twice in plates with FACS buffer. The binding of mAbs to spikes expressing on cell surface was analyzed using flow cytometry (BD) Canto Analyzer). Percent (%) PE-positive cells for antigen binding were calculated and the binding data were generated. CC12.1 (SARS-CoV-2 mAb), and CAP256. VRC26.25 antibody (HIV-1 bnAb) were used as positive and negative controls respectively for this experiment. As shown in FIG. 6, THSC20.HVTR04 and THSC20.HVTR26 found to bind more strongly than the other mAbs to the spike proteins expressed on the surface of 293T cells.

### Example 7: Pseudovirus (PSV) Neutralization Assay

Following published protocol (Science, 2020 369:956-963), pseudoviruses expressing complete SARS-CoV-2 spike genes were prepared by transient transfection of HEK293T cells with three plasmids expressing: SARS-CoV-2 spike, MLV-gag/pol and MLV-CMV-luciferase genes using Fugene 6. After 48-hour post transfection, cell supernatants containing pseudotyped viruses were harvested and frozen at −80° C. until further use. Neutralization assay was carried out using Hela-ACE2 cells for the infection of SARS-CoV-2 wild type and variant pseudoviruses. The purified IgGs were serially diluted and incubated with pseudoviruses in a humidified Incubator at 37° C. After 1-hour incubation HeLa-ACE2 cells were added to the 96-well plates at 10,000 cells/well density. After 48 hours of incubation the luciferase activity was measured by adding Britelite substrate according to manufacturer's instruction and RLU obtained using a luminometer. As shown in FIG. 7, THSC20.HVTR04 and THSC20.HVTR26 mAbs were found to potently neutralize pseudoviruses expressing spikes of Wuhan wild type, and other variants of concern; VOC (Alpha, Beta, Gamma, Delta) and variants of interest; VOI (Delta Plus, Kappa) of SARS-CoV-2 (FIGS. 7 & 8). In addition, THSC20.HVTR04 mAb showed potent neutralization of Omicron BA.2 and BA.4/BA.5 while THSC20.HVTR26 showed neutralization of Omicron BA.1 variant.

### Example 8: Live Authentic Virus Neutralization Assay

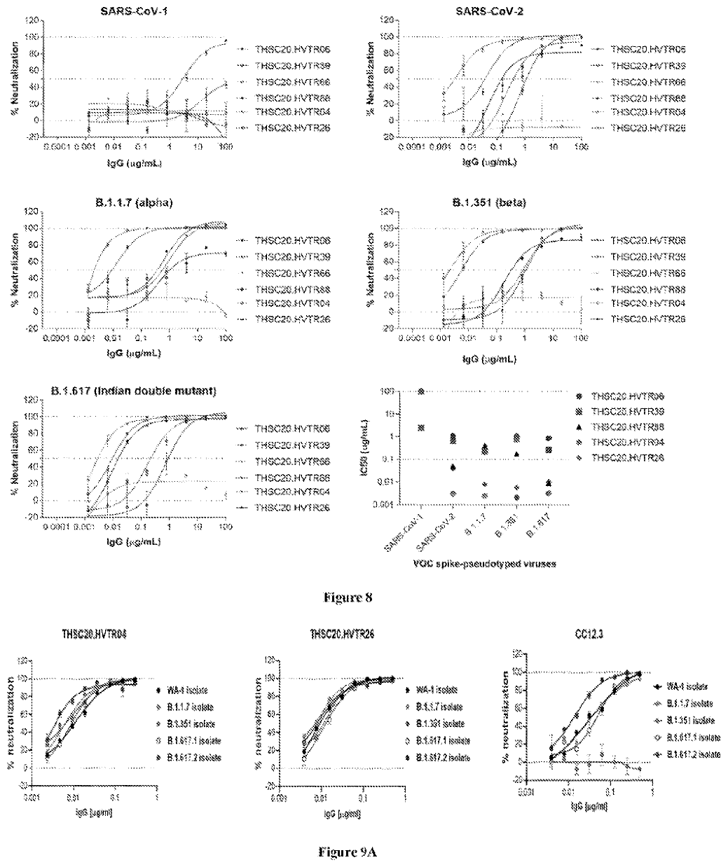

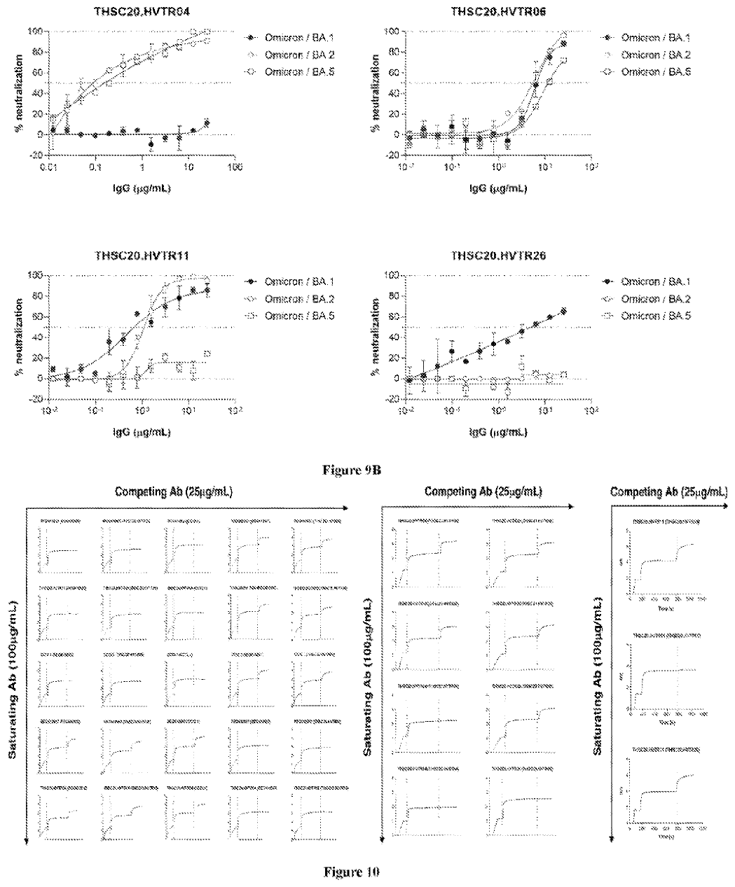

The neutralization of replication competent SARS-CoV-2 by the two novel mAbs THSC20.HVTR04 and THSC20.HVTR26 was assessed against replication competent live SARS-CoV-2 virus. The neutralization titers of both of these mAbs were next assessed by carrying out dose-dependent foci reduction neutralization test (FRNT) assay in Vero-E6 cells. As shown in FIG. 7, both THSC20.HVTR04 and THSC20.HVTR26 mAbs were found to potently neutralize SARS-CoV-2 replication competent viruses (Alpha, Beta, Kappa and Delta) with IC₅₀ of 0.01 ug/mL. In addition, THSC20.HVTR04 was found to neutralize Omicron BA.2 and BA.5 variants but not Omicron BA.1 variant and THSC20.HVTR26 demonstrated neutralization of Omicron BA.1 variant but not Omicron BA.2 and BA.5 variants. Interestingly, THSC20.HVTR06 was found to neutralize Omicron BA.1, BA.2 and BA.5 but with lower potencies, while THSC20.HVT11 was found to neutralize Omicron BA.1 and BA.2 potently but not Omicron BA.5 (FIG. 9).

### Example 9: Comparison of Epitope Specificity of THSC20.HVTR04 and THSC20.HVTR26 mAbs

All the mAbs were next assessed for epitope competition using an BLI-Octet platform. 50-100 nM of biotinylated RBD protein antigen was captured using streptavidin biosensors. After antigen loading up to wavelength shift of 1.0-1.3, a saturating concentration of mAbs (100 μg/mL) was added for 10 min. Competing concentrations of mAbs (25 μg/mL) were then added for 5 min to measure binding in the presence of saturating mAbs. All incubation steps were performed in 1×PBS. For this known mAbs to SARS-CoV-2 were used in the competition binding assay. Our data suggest that both THSC20.HVTR04 and THSC20.HVTR26 mAbs targets non-competing epitopes. (FIG. 10).

### Example 10: MAb-RBD Competition Assay

We next examined whether the isolated monoclonal antibodies compete with RBD for ACE2 receptor binding. We carried out a mAb-RBD competition for ACE2 binding assay by first incubating mAbs with RBD in a 4:1 ratio and subsequently measured binding to HeLa-ACE2 target cells by FACS. As shown in FIG. 11, among all the mAbs tested, THSC20.HVTR04 and THSC20.HVTR26 effectively blocked RBD binding to ACE2. Inhibition of SARS-CoV-2 RBD binding by mAbs to the cell surface hACE2 was assessed by flow cytometry. Briefly, purified mAbs at 100 μg/mL and biotinylated SARS-CoV-2 RBD were mixed in 100 ul of DPBS in the molar ratio of 4:1 and incubated on ice for 1 hour. Parental HeLa and HeLa-ACE2 single cell suspension were prepared by washing cells once with DPBS and then detaching by incubation with DPBS supplemented with 5 mM EDTA. The detached HeLa and HeLa-ACE2 cell suspensions were again washed once and resuspended in FACS buffer (2% FBS and 1 mM EDTA in DPBS). 0.5 million Hela-ACE2 cells were added to the test mAb/RBD mixture and then incubated at 4° C. for half an hour. 0.5 million Hela and HeLa-ACE2 cells were incubated in separate wells with RBD alone without mAbs for use as background and positive control, respectively. After washing once with PACS buffer, HeLa and HeLa-ACE2 cells were resuspended in FACS buffer containing 1 ag/ml streptavidin-PE (BD Biosciences) and incubated for another half an hour. Cells were stained with 1:1000 final dilution of LIVE/DEAD fixable aqua dead cell stain (ThermoFisher) in the same buffer for another 15 minutes. HeLa and HeLa-ACE2 cells stained with SARS-CoV-2 RBD alone were used as background and positive control separately. The PE mean fluorescence intensity (MFI) was determined from the gate of singlet and live cells and the percentage of ACE2 binding inhibition was calculated by following formula.

$$100 * ( 1 - \frac{MFI of sample - Average MFI of background}{Average MFI of probe alone - Average MFI of background} )$$

### Example 11: Pre-Clinical Efficacy of THSC20.HVTR04 and THSC20.HVTR26 mAbs in Human ACE-2 Transgenic Mouse Model

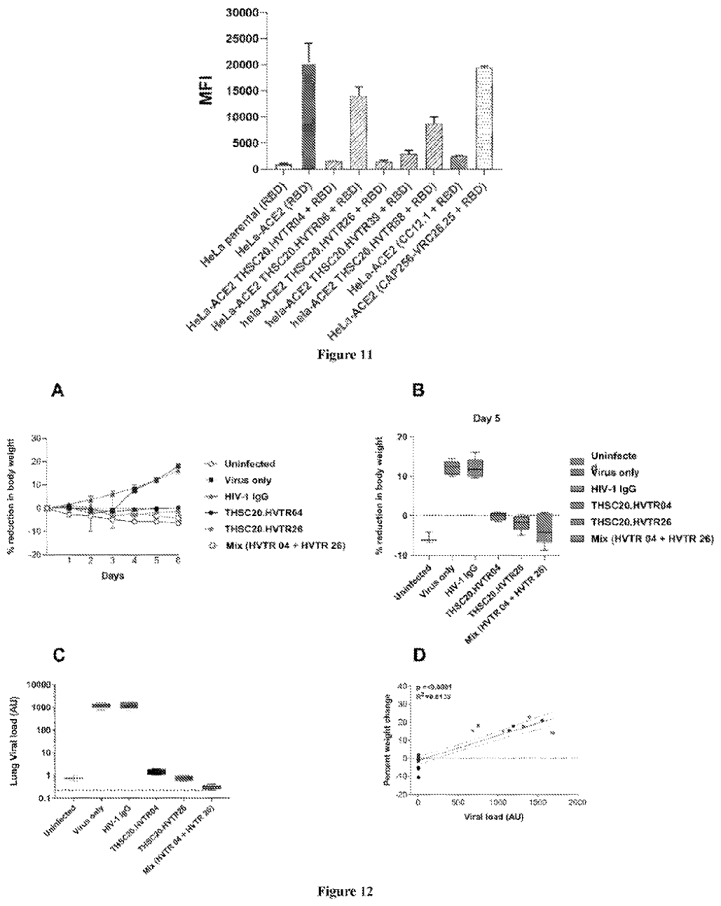

Next the prophylactic efficacy of mAbs THSC20.HVTR04 and THSC20.HVTR26 were assessed in the K18-hACE2 mouse model (Winkler et al., 2020) both independently and in combination of two mAbs. Mice were divided into six groups, each having 5 animals: (a) untreated, (b) ones those were given HIV-IgG (CAP256-VRC26.25), (c) those received mAb THSC20.HVTR04, (d) those received mAb THSC20.HVTR26 and those received the combination of THSC20.HVTR04 and THSC20.HVTR26. Each mouse under groups 2-6 received 200 μg of IgG (10 mg/kg body weight) on day before virus challenge. The body weight post virus challenge was monitored everyday till day 6. All the mice were sacrificed on day 6 and lung tissues were harvested to determine lung viral loads. All the mice in the control (not given mAbs) group and the group that received HIV-1 IgG exhibited significant loss of body weight of more than 10% at day 6, whereas no significant weight change was seen in animals who were given prophylactic injection of either of the two mAbs or their combination (FIG. 12) demonstrating significant in vivo neutralizing activity of these two mAbs. Moreover, the prophylactic activity of mAb 4 and mAb 26 was demonstrated by negligible levels of viral RNA observed in the lung tissue at 6 days post challenge of mice receiving these mAbs compared to control group animals where high level of viral RNA was seen in the lung (FIG. 12C). Overall, these results confirmed the high prophylactic efficacy of THSC20.HVTR04 and THSC20.HVTR26 against SARS-CoV-2 in the K18-HACE2 mouse model.

Both THSC20.HVTR04 and THSC20.HVTR26 that showed protection against Delta infection are not polyreactive in nature, indicating that they are suitable as products for clinical development.

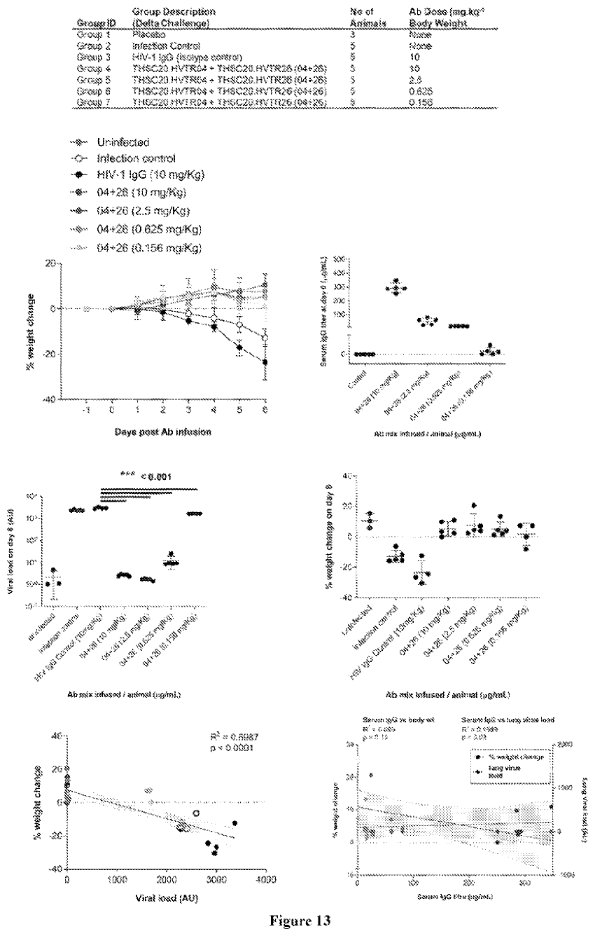

To further assess whether these two mAbs could also demonstrate protection against the more virulent Delta (B.1.617.2) variant, we repeated the same strategy as described above. As shown in FIG. 13, we arranged the mice into seven groups and titrated the combination of neutralizing antibodies to 3.125 g per animal (equivalent to 0.156 mg/kg). The results are comparable to the Wuhan challenge experiment-all the mice in the infection control group (group 2) and the isotype control group (group 3) exhibited significant weight loss at day 6, whereas no significant weight loss was observed in animals those received the antibody cocktail including those received the lowest dose (0.156 mg/kg body weight; group 7). In addition, as an indicator of clinical prognosis, the Inventors measured lung viral RNA at day 6 and found significant protection at a low antibody dose of 0.625 mg/kg body weight (P<0.001). Interestingly, the Inventors observed near 2-fold lower lung virus load in animals those received the lowest dose of the mAb cocktail (0.156 mg/kg body weight; group 7). Taken together the present invention demonstrated protection by a combination of potently neutralizing antibodies against the highly virulent Delta variant in a transgenic hACE-2 mice model.

### Example 12: Measuring Antibody Polyreactivity

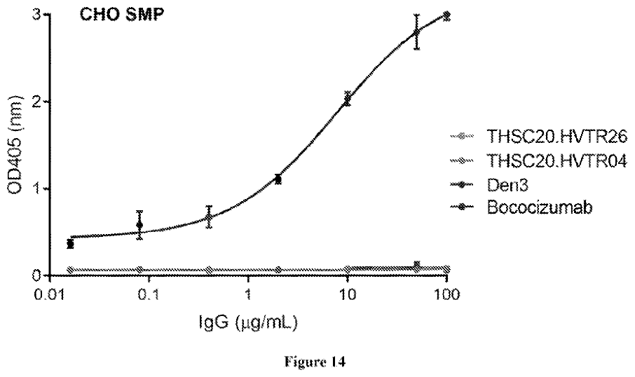

Solubilized CHO cell membrane protein (SMP) was coated onto 96-well half-area high-binding ELISA plates (Corning, 3690) at 5 ug/mL in PBS overnight at 4° C. After washing, plates were blocked with PBS/3% BSA for 1 hour at room temperature (RT). Antibodies were diluted at 100 ug/mL in 1% BSA with 5-fold serial dilution. Serially diluted samples were then added in plates and incubated for 1 hour at RT. After washing, alkaline phosphatase-conjugated goat anti-human IgG Fcy secondary antibody (Jackson ImmunoResearch, 109-055-008) was added in 1:1000 dilution and incubated for 1 h at RT. After final wash, phosphatase substrate (Sigma-Aldrich, S0942-200TAB) was added into each well. The absorption was measured at 405 nm in a spectrophotometer. Den3 (Dengue-specific mAb) and Bococizumab (PCK9 antagonist) were included as benchmarking controls. As shown in FIG. 14, both THSC20.HVTR04 and THSC20.HVHTR26 were found to be non-polyreactive indicating these mAbs could be taken for clinical development.

We claim:

1. Composition of seven novel human monoclonal antibodies which exhibits strong binding to receptor binding domain of the viral spike protein of SARS-CoV-2, comprises:
a) THSC20.HVTR04 comprising variable Heavy chain IgG sequence of SEQ ID NO. 1 and variable light chain IgG sequence of SEQ ID NO. 2
b) THSC20.HVTR06 comprising variable heavy chain IgG sequence of SEQ ID NO. 3 and variable light chain IgG sequence of SEQ ID NO. 4
c) THSC20.HVTR11 comprising variable heavy chain IgG sequence of SEQ ID NO. 5 and variable light chain IgG sequence of SEQ ID NO. 6
d) THSC20.HVTR26 comprising variable heavy chain IgG sequence of SEQ ID NO. 7 and variable light chain IgG sequence of SEQ ID NO. 8
e) THSC20.HVTR39 comprising variable heavy chain IgG sequence of SEQ ID NO. 8 and variable light chain IgG sequence of SEQ ID NO. 9
f) THSC20.HVTR55 comprising variable heavy chain IgG sequence of SEQ ID NO. 11 and variable light chain IgG sequence of SEQ ID NO. 12
g) THSC20.HVTR88 comprising variable heavy chain IgG sequence of SEQ ID NO. 13 and variable light chain IgG sequence of SEQ ID NO. 14

2. The composition as claimed in claim 1, wherein the monoclonal antibodies neutralizes SARS-CoV-2 and its variants.

3. The composition as claimed in claim 1, wherein the panel comprises THSC20.HVTR04, THSC20.HVTR06, THSC20.HVTR11, THSC20.HVTR26, THSC20.HVTR39, THSC20.HVTR55 and THSC20.HVTR88.

4. The composition as claimed in claim 1, wherein the antibodies are human monoclonal antibodies.

5. The composition as claimed in claim 1, wherein the monoclonal antibodies neutralizes pseudoviruses expressing spikes of Wuhan strain, Delta variant (B.1.617.2), UK variant or Alpha (B.1.1.7), South African variant or Beta (B.1.351), Brazilian variant or Gamma (P.1), Kappa (B.1.617.1), Delta (B.1.617.2) Delta Plus variant.

6. The composition as claimed in claim 1, comprises novel variable and light IgG chain sequences obtained from a single unvaccinated but infected individual and originated from unique B cell germline genes.

7. The composition as claimed in claim 1, wherein the live authentic virus neutralization of key mAbs comprises THSC20.HVTR04, THSC20.HVTR26 against Wuhan, Alpha, Beta, Kappa and THSC20.HVTR04, THSC20.HVTR06, THSC20.HVTR11, THSC20.HVTR26 against Omicron variants (BA.1, BA.2 and BA.5).

8. The composition as claimed in claim 1, wherein THSC20.HVTR04, and THSC20.HVTR26 provides protection of hACE-2 K18 mice against Wahan and Delta isolates.

9. A therapeutic composition comprising combination of THSC20.HVTR04 and THSC20.HVTR26 mAbs against SARS-CoV-2 Delta variant wherein the therapeutic composition is present in an amount of 0.625 mg/kg body weight.

10. A method for isolating monoclonal antibodies (mAbs) against SARS-CoV-2 as claimed in claim 1, comprising the steps of:
a) selection of a donor with high neutralization titer of antibodies in the plasma;
b) Sorting of SARS-CoV-2 specific single IgG positive B cells using biotinylated SARS-CoV-2 RBD protein as an antigen, which were subsequently used as source for amplification of heavy and light chain variable genes of IgG from single B cell
c) Novel broadly neutralizing antibodies (bnAbs) were obtained by emphasizing neutralization as the initial screen.

11. A method for obtaining the monoclonal antibodies (mAbs) as claimed in claim 1, comprising the steps of:
i) Sorting of SARS-CoV-2 specific single B cells from a donor PBMC sample for neutralization activity against a plurality of SARS-CoV-2 variants
ii) RT-PCR and amplification of variable heavy and light IgG sequences
iii) Cloning of heavy and light chain variable functional antibody genes from a single B cell that exhibits neutralization activity tested by pseudovirus and live virus neutralization assays
iv) Selection of the desired mAb clones and scaling up for IgG purification by co-transfection of plasmid DNA expressing variable heavy and light chain IgG sequences in Expi-293 cells.


SEQUENCE LISTING (14 sequences)

SEQ ID NO: 1 - DNA, 370 bp, synthetic construct
  source 1..370: mol_type = other DNA
caggtgcagctggtggagtctgggggaggcgtggtccagcctgggaggtccctgagactctcctgtgcagcctctggattcaccttcagtaactatgctatccactgggtccgccaggctccaggcaaggggctggagtgggtggcagttgtgtcatatgatggaagcaataaatactacgcagagtccgtgaagggccgattcaccatctccagagacaattccaagaacacgctgtctctacaaatgatcagcctgagacctgaggacacggctgtgtattactgtgcgagcgtcgcggatacagctatggttccagagtggtacttcgatctctggggccgtggcaccctggtcaccgtctcttcag

SEQ ID NO: 2 - DNA, 325 bp, synthetic construct
  source 1..325: mol_type = other DNA
cagtctgtgctgactcagcctcgctcagtgtccgggtctcctggacagtcagtcaccatctcctgcactggaaccagcagtgatgttggtgcttataactatgtctcctggtaccaacagcacccaggcaaagcccccaaactcatgatttatgatgtcagtgcgcggccctcaggggtccctgatcgcttctctggctccaagtctggcaacacggcctccctgaccatctctgggctccaggctgaggatgaggctgattattactgctgctcatatgccggcagctgggtgttcggcggagggaccaagctgaccgtcctag

SEQ ID NO: 3 - DNA, 390 bp, synthetic construct
  source 1..390: mol_type = other DNA
cagctgcagctggtgcaatctgggtctgagttgaagaagcctggggcctcagtgaaggtttcctgcaaggcttctggatacaccttcactaagtatactgtaaattgggtgcgacaggcccctggacaagggcttgagtggatgggatggatcaacaccaacactgggaacccaacgtctgcccagggcttcacaggacggtttgtcttctccttggacacctctgtcagcacggcatatctgcacatcagcagcctaaagactgaagacactgccgtgtattactgtgcgagggtgttaggggaccagcaggtggtgggaggcgacgattactactactactacgctatggacgtctggggccaagggaccccggtcaccgtctcctca

SEQ ID NO: 4 - DNA, 319 bp, synthetic construct
  source 1..319: mol_type = other DNA
tcctatgagctgactcagccaccctcagtgtccgtgtccccaggacagacagccagcatcacctgctctggagataaattgggggataaatatgcttcctggtatcagcagaagccaggccagtcccctgtattggtcatctatcaagataccaagcggccctcagggatccctgagcgattctctggctccaactctgggaacacagccactctgaccatcaccgggacccaggctatggatgagactgactattactgtcaggcgtgggacaacagcggctgggtattcggcggagggaccaagctgaccgtcctag

SEQ ID NO: 5 - DNA, 373 bp, synthetic construct
  source 1..373: mol_type = other DNA
gaggtgcagctggtgcaatctggggctgaggtgaagaagcctgggtcctcggtgaaggtctcctgcaaggcttctggaggcaccttcagcagttatactatcagctgggtgcgacaggcccctggacaagggcttgagtggatgggaaggatcatccccatccttggtatagcaaactacgcacagaagttccaaggcagagtcacgattaccgcggacaaatccacgagcacagcctgcatggagctgagcagcctgagatctgaggacacggccgtgtatttctgtgcgaggatacgcggatatagtggctacggttcttcgtactactttgactactggggccagggaaccctggtcaccgtctcctcag

SEQ ID NO: 6 - DNA, 334 bp, synthetic construct
  source 1..334: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagtttgagccatgaggtattcggcggagggaccaaactgaccgtcctag

SEQ ID NO: 7 - DNA, 382 bp, synthetic construct
  source 1..382: mol_type = other DNA
gaggtgcagctggtggagtctggaggaggcttggtccagcctggggggtccctgagactctcctgtgcagcctctgggttcaccgtcagtagcaactacatgagctgggtccgccaggctccagggaaggggctggagtgggtctcagctatttatagcggggatagtacatactacgcagactccgtgaagggccgattcaccatctccagacacaatcccaagaacacgctgtatcttcaaatgaacagcctgagagctgaggacacggccgtgtattactgtgcgagactcgtcggtgccctaacaaatatagtagtgtctggagacgggggtgcttttgatatctggggccaagggacaatggtcaccgtctcttcag

SEQ ID NO: 8 - DNA, 331 bp, synthetic construct
  source 1..331: mol_type = other DNA
tcctatgagctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgaggtcagtaagcggccctcaggggtttctaatcgcttctctggctccaagtctggcaacacggcctccctgacaatctctgggctccaggctgaggacgaggttgattattactgctgctcatatgcaggtagtagcacttgggtgttcggcggagggaccaaactgaccgtcctag

SEQ ID NO: 9 - DNA, 386 bp, synthetic construct
  source 1..386: mol_type = other DNA
ccaggtgcagctgcaggagtcgggcccaggactggtgaggccttcggagaccctgtccctcacctgcattgtctctggtggctccatcaacagtaggagttactcctggggctggatacgccagcccccagggaaggggctggagtggattggaaatgtctactatagtgggaccacctattataacccgtccctcaagagtcgagtcaccatatcagtagacacgtccaagaaccagttctccctgaacctgagctctgtgaccgccgcggacacggccgtctattactgtgcgagaaatttattttattatgatagaagtggttcctctcaccatgatgcttttgatatctggggccaagggacaatggtcaccgtctcctcag

SEQ ID NO: 10 - DNA, 318 bp, synthetic construct
  source 1..318: mol_type = other DNA
tccacggttatgtgcgcccgctgtcagtggccccaggacagacggccaggattacctgtgggggaaacaacattggaagtaaaagtgtacactggtaccagcagaagccaggccaggcccctgtgctggtcgtctatggtgatggcgaccggccctcagggatccctgagcgattctctggctccaactctggaaacacggccaccctgaccatcagcagggtcgaagccggggatgaggccgactattactgtcaggtgtgggatagaggtagtgatgtttgggtgttcggcggagggaccaagctgaccgtcctag

SEQ ID NO: 11 - DNA, 361 bp, synthetic construct
  source 1..361: mol_type = other DNA
gaggtgcagctggtgcagtctggagcagaggtgaaaaagcccggggagtctctgaggatctcctgtaagggttctggattcaggtttaccaattactggatcggctgggtgcgccagatgcccgggaaaggcctggagtggatgggtatcatctatcctggtgactctgataccagatacagcccgtccttccaaggccaggtcaccatctcagccgacaagtccctcagcaccgcctacctgcagtggagcagcctgcaggcctcggacaccgccatctattactgtgcgagacgggatcggtgggagctagatcctcttgactactggggccagggaaccctggtcaccgtctcctcag

SEQ ID NO: 12 - DNA, 322 bp, synthetic construct
  source 1..322: mol_type = other DNA
gacatcgtgatgacccagtctccttccaccctgtctgcatctgtaggagacagagtcaccatcacttgccgggccagtcagagtattagtagctggttggcctggtatcagcagaaaccagggaaagcccctaacctcctgatctataaggcgtctagtttagaaagtggggtcccatcaaggttcagcggcagtggatctgggacagaattcactctcaccatcagcggcctgcagcctgatgattttgcaacttattactgccaacactataatggttacccctacacttttggccaggggaccaagctggagatcaaac

SEQ ID NO: 13 - DNA, 376 bp, synthetic construct
  source 1..376: mol_type = other DNA
caggtgcagctggtgcagtctggagctgaggtgaagaagcctggggcctcagtgaaggtctcctgcaaggcttctggttacccctttaccagctatgttatcagctgggtgcgacaggcccctggacaagggcttgagtggatgggatggatcagcgcttacaatggtaacacagactatgcacagaagctccagggcagagtcaccatgaccacagacacatccacgagcacagcctacatggagctgaggagcctgagatctgacgacacggccgtgtattactgtgcgagagaggggaagtatagcagtggctggtcttatagtgaatactttgactactggggccagggaaccctggtcaccgtctcttcag

SEQ ID NO: 14 - DNA, 325 bp, synthetic construct
  source 1..325: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactttggacgttcggccaagggaccaagctggagatcaaac